(19) United States
(12) Patent Application Publication
(10) Pub. No.: US 2018/0021320 A1
(43) Pub. Date: Jan. 25, 2018

(54) Method of Preventing or Treating Hearing Loss

(71) Applicant: SUPPORT-VENTURE GMBH, Basel (CH)

(72) Inventor: Alexander BAUSCH, Riehen (CH)

(21) Appl. No.: 15/550,268

(22) PCT Filed: Feb. 10, 2016
(86) PCT No.: PCT/EP2016/052787

§ 371 Date: Aug. 10, 2017

(30) Foreign Application Priority Data
Feb. 11, 2015 (EP) 15154764.3

(51) Int. Cl. A61K 31/4439; A61K 9/00

(52) U.S. Cl. CPC A61K 31/4439; A61K 9/0046

(57) ABSTRACT

The present invention relates to methods of preventing or treating hearing loss and methods of preventing or inhibiting hair cell degeneration or hair cell death in a subject.

### THE FIELD OF THE INVENTION

The present invention relates to methods of preventing or treating hearing loss and methods of preventing or inhibiting hair cell degeneration or hair cell death in a subject.

### BACKGROUND OF THE INVENTION

Hearing loss is related to damage of the hair cells e.g. apoptosis of the hair cells as a consequence of e.g. a continuous stress situation or a traumatic event e.g. leading to the activation of inflammatory pathways. Hearing loss can be caused e.g. by a noise trauma, by a medical intervention, by ischemic injury, by a non specific stress leading to sudden hearing loss or by age or can be chemically induced, wherein the chemical induction is caused e.g. by an antibiotic or a chemotherapeutic agent. Child hearing loss might be caused by pre or post natal deficiencies in the energy homeostasis of the neural cells. Hearing loss can also be caused by mitochondrial dysfunction. (C. M. Sue PhD, FRACP1, Cochlear origin of hearing loss in MELAS syndrome, Annals of Neurology. Volume 43, Issue 3, pages 350-359, March 1998). In addition a link between metabolic syndrome and hearing loss could be shown (Barrenäs M L, Jonsson B, Tuvemo T, Hellström P A, Lundgren M, J Clin Endocrinol Metab. 2005 August; 90(8):4452-6. Epub 2005 May 31). Hearing loss can be of sensorineural origin caused by a damage leading to malnutrition of the cells in early brain development.

Hair cells are fully differentiated and are not replaced after cell death (only a few thousand cells from birth). It is well described in the literature that after stress and damage of the hair cells, the cells can go in a resting state with no functionality related to the hearing process but remaining viable in a resting state. Approaches to stimulate development or regeneration of new hair cells e.g. by administering growth factors or by stem cell-based therapies in order to achieve disease modification bear the risk of pro-tumorigenic side-effects.

Hearing impairment is a major global health issue with profound societal and economic impact affecting over 275 million people world-wide. The occurrence of hearing loss is rapidly rising, due to e.g. increasing noise exposure and aging populations. With no approved pharmaceutical therapies available today, the unmet medical need is very high. In particular there is a need for providing effective methods for prevention and subsequent treatment of hearing loss which allow for immediate as well as long term maintenance of preventive and/or therapeutic effects.

### SUMMARY OF THE INVENTION

The present invention relates generally to methods of preventing or treating hearing loss and methods of preventing or inhibiting hair cell degeneration or hair cell death using a PPAR agonist. The present invention provides methods which allow for protection of hair cells from stress e.g. from noise induced stress or from chemically induced stress such as stress induced by an antibiotic or by a chemotherapeutic agent or from unspecific stress which may cause hearing loss. Using the methods described herein, immediate and subsequent long term maintenance of preventive and/or therapeutic effect can be achieved. In a standard model established in hearing loss research, it could be shown that treatment with a PPAR agonist protects hair cells, which upon exposure to an antibiotic are normally destroyed within 48 hours. The addition of the PPAR agonist prior to antibiotic challenge was able to prevent hair cells from apoptosis and cell death in a dose-dependent manner. Without limitation to theory, it is assumed that the prevention or treatment of hearing loss and/or the prevention or inhibition of hair cell degeneration or hair cell death is achieved by one or more, or a combination of the following pathway interactions: by reducing oxidative stress and/or by down-regulation of the MAPK pathway via prevention of JNK phosphorylation and/or by restoring insulin sensitivity via the IRS1 pathway, AKT pathway, GLUT4 pathway or the GSK3 pathway, and/or by restoring ribosomal functionality, and/or by improving mitochondrial content or functionality.

In a first aspect, the present invention relates to a PPAR agonist for use in a method of preventing or treating hearing loss in a subject.

In a further aspect, the present invention relates to a PPAR agonist for use in a method of preventing or inhibiting hair cell degeneration or hair cell death in a subject.

In still another aspect the present invention relates to a pharmaceutical composition comprising a PPAR agonist and a pharmaceutically acceptable diluent, excipient, or carrier for use in a method of preventing or treating hearing loss in a subject.

In a further aspect, the present invention relates to a kit for preventing or treating hearing loss or preventing or inhibiting hair cell degeneration or hair cell death in a subject comprising a PPAR agonist.

### BRIEF DESCRIPTION OF THE FIGURES

FIG. 1A-C show quantitation of the average number of hair cells remaining in the apical, basal and middle turns of the organ of Corti (OC). While gentamicin (200 μM) treatment resulted in a consistent reduction of hair cell number of approximately 50-70% in each segment, Pioglitazone at both concentrations (2 μM and 10 μM) was able to significantly prevent gentamicin-dependent hair cell loss in all turns. The values for each turn were averaged for the 10 OCs used for each condition. Significant differences between treatment groups in OHC and IHC (OHC=outer hair cell; IHC=inner hair cell) were determined using analysis of variance (ANOVA) followed by the least significant difference (LSD) post-hoc test (Stat View 5.0). Differences associated with P-values of less than 0.05 were considered to be statistically significant. All data are presented as mean±SD.

FIG. 2 shows the change in the average hearing thresholds in guinea pigs determined by auditory brainstem response (ABR) one week or two weeks after noise challenge vs. pre-treatment values. Threshold shifts at individual frequencies were calculated for each animal by subtracting post-noise from pre-noise values. Group averages at each frequency were determined. An overall threshold shift was calculated for each treatment group and timepoint by averaging the individual frequency shifts over 8-20 KHz. Data are mean±S.D. *p<0.05.

FIG. 3A-C show quantitation of the average number of hair cells remaining in defined segments in the medio-basal turns of the organ of Corti (OC). While gentamicin (50 μM) treatment resulted in a consistent reduction of hair cell number of approximately 50%, tesaglitazar, muraglitazar and fenofibric acid were all able to significantly prevent gentamicin-dependent hair cell loss. The values were averaged for the 5-7 OCs used for each condition. Significant differences between treatment groups in hair cell numbers were determined using analysis of variance (ANOVA) followed by the least significant difference (LSD) post-hoc test (Stat View 5.0). Differences associated with P-values of less than 0.05 were considered to be statistically significant. All data are presented as mean±SD. ****=p≦0.001.

### DETAILED DESCRIPTION OF THE INVENTION

The present invention provides methods of preventing or treating hearing loss and methods of preventing or inhibiting hair cell degeneration or hair cell death.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting. The terms “comprising”, “having”, and “including” are to be construed as open-ended terms (i.e., meaning “including, but not limited to”) unless otherwise noted.

The term “ PPAR agonist” as used herein refers to a drug that is activating peroxisome proliferator activated receptor (PPAR) such as PPAR gamma receptor, PPAR alpha receptor, PPAR delta receptor or combinations thereof and includes PPAR gamma agonists such as e.g. pioglitazone, troglitazone or rosiglitazone, PPAR alpha agonists such as e.g. fibrates such as fenofibrate (fenofibric acid), clofibrate or gemfibrozil, PPAR dual agonists (PPAR alpha/gamma or PPAR alpha/delta agonists) such as e.g. aleglitazar, muraglitazar, tesaglitazar, ragaglitazar, saroglitazar, GFT505 or naveglitazar, PPAR delta agonists such as e.g. GW501516, PPAR pan agonists (PPAR alpha/delta/gamma agonist) or selective PPAR modulators such as e.g. INT131 and the salts of these compounds. Usually PPAR gamma agonists, PPAR modulators, PPAR alpha agonists and/or PPAR alpha/gamma dual agonists are used in the methods of the present invention, in particular PPAR gamma agonists, PPAR alpha agonists and/or PPAR alpha/gamma dual agonists are used in the methods of the present invention, more particular PPAR gamma agonists selected from the group consisting of pioglitazone, rosiglitazone, troglitazone, preferably pioglitazone, PPAR alpha agonists selected from the group consisting of fenofibrate (fenofibric acid), clofibrate and gemfibrozil, preferably fenofibrate (fenofibric acid) and/or PPAR alpha/gamma dual agonists selected from the group consisting of aleglitazar, muraglitazar, tesaglitazar, ragaglitazar, saroglitazar, GFT505 and naveglitazar, preferably muraglitazar or tesaglitazar. Preferably PPAR gamma agonists are used in the methods of the present invention, more preferably PPAR gamma agonists or modulators selected from the group consisting of pioglitazone, rosiglitazone, troglitazone, INT131, even more preferably PPAR gamma agonists selected from the group consisting of pioglitazone, rosiglitazone and troglitazone are used. Most preferably pioglitazone or its salts e.g. pioglitazone hydrochloride is used. Pioglitazone is described e.g. in U.S. Pat. No. 4,687,777 or in Dormandy J A, Charbonnel B, Eckland D J, Erdmann E, Massi-Benedetti M, Moules I K, Skene A M, Tan M H, Lefèbvre P J, Murray G D, Standl E, Wilcox R G, Wilhelmsen L, Betteridge J, Birkeland K, Golay A, Heine R J, Korányi L, Laakso M, Mokán M, Norkus A, Pirags V, Podar T, Scheen A, Scherbaum W, Schernthaner G, Schmitz O, Skrha J, Smith U, Taton J; PROactive investigators. Lancet. 2005 Oct. 8; 366(9493):1279-89, and is represented by the structural formula indicated below:

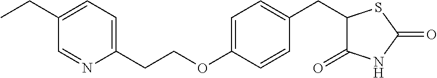 [SMILES: CCc1ccc(CCOc2ccc(CC3SC(=O)NC3=O)cc2)nc1]

Troglitazone is described e.g. in Florez J C, Jablonski K A, Sun M W, Bayley N, Kahn S E, Shamoon H, Hamman R F, Knowler W C, Nathan D M, Altshuler D; Diabetes Prevention Program Research Group. J Clin Endocrinol Metab. 2007 April; 92(4):1502-9 and is represented by the structural formula indicated below:

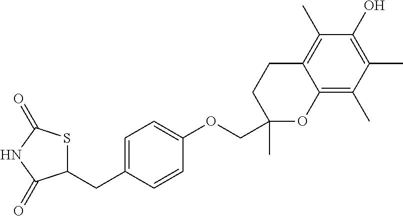 [SMILES: Cc1c(C)c2c(c(C)c1O)CCC(C)(COc1ccc(CC3SC(=O)NC3=O)cc1)O2]

Rosiglitazone is described e.g. in Nissen S E, Wolski K. N Engl J Med. 2007 Jun. 14; 356(24):2457-71. Erratum in: N Engl J Med. 2007 Jul. 5; 357(1):100. Fenofibrate is described e.g. in Bonds D E, Craven T E, Buse J, Crouse J R, Cuddihy R, Elam M, Ginsberg H N, Kirchner K, Marcovina S, Mychaleckyj J C, O'Connor P J, Sperl-Hillen J A. Diabetologia. 2012 June; 55(6):1641-50 and is represented by the structural formula indicated below:

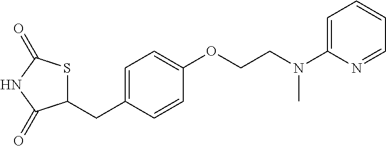 [SMILES: CN(CCOc1ccc(CC2SC(=O)NC2=O)cc1)c1ccccn1]

Clofibrate is described e.g. in Rabkin S W, Hayden M, Frohlich J. Atherosclerosis. 1988 October; 73(2-3):233-40 and is represented by the structural formula indicated below:

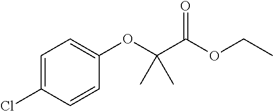 [SMILES: CCOC(=O)C(C)(C)Oc1ccc(Cl)cc1]

Fenofibrate (fenofibric acid) is described e.g. in Schima S M, Maciejewski S R, Hilleman D E, Williams M A, Mohiuddin S M. Expert Opin Pharmacother. 2010 April; 11(5):731-8 and is represented by the structural formula indicated below:

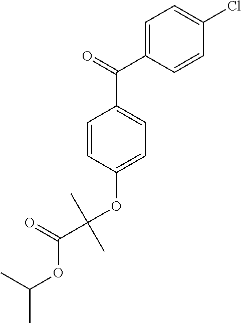 [SMILES: CC(C)OC(=O)C(C)(C)Oc1ccc(C(=O)c2ccc(Cl)cc2)cc1]

Gemfibrozil is described e.g. in Adabag A S, Mithani S, Al Aloul B, Collins D, Bertog S, Bloomfield H E; Veterans Affairs High-Density Lipoprotein Cholesterol Intervention Trial Study Group. Am Heart J. 2009 May; 157(5):913-8 and is represented by the structural formula indicated below:

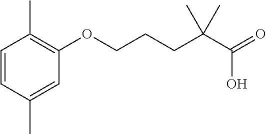 [SMILES: Cc1ccc(C)c(OCCCC(C)(C)C(=O)O)c1]

Aleglitazar is described e.g. in Lincoff A M, Tardif J C, Schwartz G G, Nicholls S J, Rydén L, Neal B, Malmberg K, Wedel H, Buse J B, Henry R R, Weichert A, Cannata R, Svensson A, Volz D, Grobbee D E; AleCardio Investigators. JAMA. 2014 Apr. 16; 311(15):1515-25 and is represented by the structural formula indicated below:

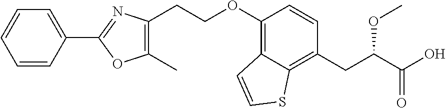 [SMILES: CO[C@@H](Cc1ccc(OCCc2nc(-c3ccccc3)oc2C)c2ccsc12)C(=O)O]

Muraglitazar is described e.g. in Fernandez M, Gastaldelli A, Triplitt C, Hardies J, Casolaro A, Petz R, Tantiwong P, Musi N, Cersosimo E, Ferrannini E, DeFronzo R A. Diabetes Obes Metab. 2011 October; 13(10):893-902 and is represented by the structural formula indicated below:

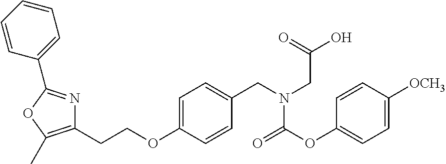 [SMILES: COc1ccc(OC(=O)N(CC(=O)O)Cc2ccc(OCCc3nc(-c4ccccc4)oc3C)cc2)cc1]

Tesaglitazar is described e.g. in Bays H, McElhattan J, Bryzinski B S; GALLANT 6 Study Group. Diab Vasc Dis Res. 2007 September; 4(3):181-93 and is represented by the structural formula indicated below:

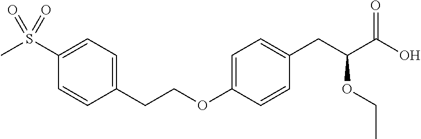 [SMILES: CCO[C@@H](Cc1ccc(OCCc2ccc(S(C)(=O)=O)cc2)cc1)C(=O)O]

Ragaglitazar is described e.g. in Saad M F, Greco S, Osei K, Lewin A J, Edwards C, Nunez M, Reinhardt R R; Ragaglitazar Dose-Ranging Study Group. Diabetes Care. 2004 June; 27(6):1324-9 and is represented by the structural formula indicated below:

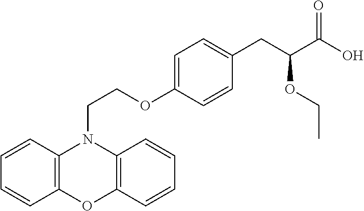 [SMILES: CCO[C@@H](Cc1ccc(OCCN2c3ccccc3Oc3ccccc32)cc1)C(=O)O]

Saroglitazar is described e.g. in Agrawal R. Curr Drug Targets. 2014 February; 15(2):151-5 and is represented by the structural formula indicated below:

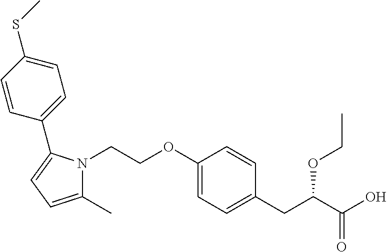 [SMILES: CCO[C@@H](Cc1ccc(OCCn2c(C)ccc2-c2ccc(SC)cc2)cc1)C(=O)O]

Naveglitazar is described e.g. in Ahlawat P, Srinivas N R. Eur J Drug Metab Pharmacokinet. 2008 July-September; 33(3):187-90. GW501516 is described e.g. in Wang X, Sng M K, Foo S, Chong H C, Lee W L, Tang M B, Ng K W, Luo B, Choong C, Wong M T, Tong B M, Chiba S, Loo S C, Zhu P, Tan N S. J Control Release. 2015 Jan. 10; 197:138-47 and is represented by the structural formula indicated below:

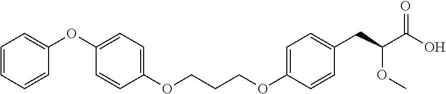 [SMILES: CO[C@@H](Cc1ccc(OCCCOc2ccc(Oc3ccccc3)cc2)cc1)C(=O)O]

GFT505 is described e.g. in Cariou B, Staels B. Expert Opin Investig Drugs. 2014 October; 23(10):1441-8 and is represented by the structural formula indicated below:

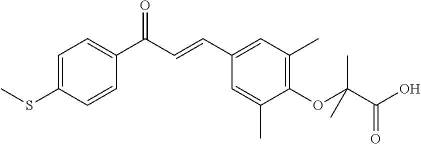 [SMILES: CSc1ccc(C(=O)/C=C/c2cc(C)c(OC(C)(C)C(=O)O)c(C)c2)cc1]

INT131 is described e.g. in Taygerly J P, McGee L R, Rubenstein S M, Houze J B, Cushing T D, Li Y, Motani A, Chen J L, Frankmoelle W, Ye G, Learned M R, Jaen J, Miao S, Timmermans P B, Thoolen M, Kearney P, Flygare J, Beckmann H, Weiszmann J, Lindstrom M, Walker N, Liu J, Biermann D, Wang Z, Hagiwara A, Iida T, Aramaki H, Kitao Y, Shinkai H, Furukawa N, Nishiu J, Nakamura M. Bioorg Med Chem. 2013 Feb. 15; 21(4):979-92 and is represented by the structural formula indicated below:

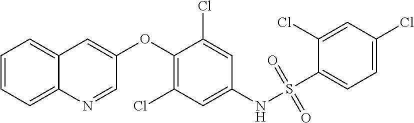 [SMILES: O=S(=O)(Nc1cc(Cl)c(Oc2cnc3ccccc3c2)c(Cl)c1)c1ccc(Cl)cc1Cl]

PPAR activation by the PPAR agonist is usually strong in the low nanomolar range to low micromolar range, e.g in a range of 0.1 nM to 100 μM. In some embodiments the PPAR activation is weak or partial, i.e. a PPAR agonist is used in the methods of the present invention which yields maximal activation of PPAR-receptor in a reporter assay system of 10% to 100% compared to a reference PPAR agonist which is known to causes a maximum PPAR activation. The preferred target for interaction of the PPAR agonist is the hair cell, which is most preferred, neural cells, and endothelial cells, and further includes adipocytes, hepatocytes, immune cells such as e.g. macrophages or dendritic cells, or skeletal muscle cells.

The term “hearing loss” which is used herein interchangeably with the term “hearing impairment” refers to a diminished sensitivity to the sounds normally heard by a subject. The severity of a hearing loss is categorized according to the increase in volume above the usual level necessary before the listener can detect it. The term “hearing loss” as used herein includes sudden hearing loss (SHL) which is indicated in the literature also as “sudden sensorineural hearing loss (SSHL)”. SHL refers to illness which is characterized by a sudden, rapid sensorineural hearing loss mostly in one ear without obvious causes, normally accompanied with dizziness, and without vestibular symptomatology. SHL is defined as greater than 30 dB hearing reduction, over at least three contiguous frequencies, occurring over a period of 72 hours or less. SHL can be caused e.g. by unspecific stress.

Hearing loss as referred herein is defined as a diminished ability to hear sounds like other people do. This can be caused either by conductive hearing loss, sensorineural hearing loss or a combination of both.

Conductive hearing loss means that the vibrations are not passing through from the outer ear to the inner ear, specifically the cochlea. It can be due to an excessive build-up of earwax, glue ear, an ear infection with inflammation and fluid buildup, a perforated eardrum, or a malfunction of the ossicles (bones in the middle ear). Also, the eardrum may be defective. Sensorineural hearing loss is caused by dysfunction of the inner ear, the cochlea, auditory nerve, or brain damage. Usually, this kind of hearing loss is due to damage of the hair cells in the cochlea.

Hearing loss as referred herein is usually sensorineural hearing loss or a combination of conductive hearing loss and sensorineural hearing loss. Sensorineural hearing loss can be related to age, to an acute or constant exposure to noise or chemicals, to a brain trauma or non specific stress which may lead to sudden hearing loss.

The term “hair cell degeneration” as used herein refers to a gradual loss of hair cell function and integrity and/or leading ultimately to hair cell death.

The term “hair cell death” as used herein refers to apoptosis of the hair cells in the inner ear.

The terms “identification of hair cell damage” or “detection of hair cell damage” which are used interchangeably herein refer to a method by which the degree of hair cell damage in the inner ear can be determined. Such methods are known in the art and comprise for example fluorescent imaging of the hair cells, as described in the examples. An audiogram that demonstrates loss of hearing sensitivity at moderate to high frequencies is also indicative of hair cell damage. A decrease of hearing potential with no subsequent recovery is also diagnostic of hair cell damage.

The term “chemically induced hearing loss” or “hearing loss induced by a chemical” as referred herein refers to hearing loss which is induced and/or caused by chemical agents such as solvents, gases, paints, heavy metals, and/or medicaments which are ototoxic.

The term sound pressure level (SPL) or acoustic pressure level as referred herein is a logarithmic measure of the effective sound pressure of a sound relative to a reference value. Sound pressure level, denoted Lₚ and measured in dB, above a standard reference level, is given by:

Lₚ=10 log₁₀ (pᵣₘₛ²/p₀²)=20 log₁₀ (pᵣₘₛ/p₀) dB(SPL)

where pᵣₘₛ is the root mean square sound pressure, measured in Pa and p₀ is the reference sound pressure, measured in Pa. The commonly used reference sound pressure in air is p₀=20 μPa (Root Mean Squared) or 0.0002 dynes/cm², which is usually considered the threshold of human hearing.

The term “pharmaceutically acceptable carrier” as used herein refers to a carrier or excipient or diluent that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The term “individual,” “subject” or “patient” are used herein interchangeably. In certain embodiments, the subject is a mammal. Mammals include, but are not limited to primates (including human and non-human primates). In a preferred embodiment, the subject is a human.

The term “about” as used herein refers to +/−5% of a given measurement.

In one aspect, the present invention provides a PPAR agonist for use in a method of preventing or treating hearing loss in a subject. In a further aspect of the invention the present invention provides a method of preventing or treating hearing loss in a subject, which method comprises administering to the subject a PPAR agonist. In some embodiments the PPAR agonist is administered to the subject in an amount that is sufficient to prevent or treat hearing loss in the subject. In a further aspect the present invention provides the use of a PPAR agonist for the manufacture of a medicament for preventing or treating hearing loss in a subject.

In some preferred embodiments hearing loss to be prevented or treated by the methods of the present invention is caused by a noise trauma, by a medical intervention, by ischemic injury, by age or is chemically induced. The hearing loss can be thus a consequence of a medical intervention such as e.g. cochlear implantation. The chemical induction is usually caused by a chemical agent e.g. by an antibiotic or a chemotherapeutic agent. In some preferred embodiments hearing loss is sudden hearing loss. Hearing loss caused by age comprises e.g. presbycusis. Preferably hearing loss caused by a noise trauma, cochlear implantation, or which is chemically induced, preferably by an antibiotic, is prevented or treated by the methods of the present invention. More preferably hearing loss caused by a noise trauma or which is chemically induced, preferably by an antibiotic, is prevented or treated by the methods of the present invention. In some embodiments, hearing loss is of sensorineural origin caused by a damage leading to malnutrition of the cells in early brain development. In this case early treatment with a PPAR agonist can be disease modifying preventing further damage.

In some embodiments the PPAR agonist is administered before the subject has developed or before it is at risk to develop hearing loss, hair cell degeneration, hair cell death and/or a condition characterized by hair cell damage. In some embodiments, the PPAR agonist is administered after the subject has acquired hearing loss, hair cell degeneration, hair cell death and/or a condition characterized by hair cell damage.

Further diseases, disorders or conditions which are related to, caused or characterized by hair cell degeneration and/or hair cell death and which can be prevented or treated by the methods of the present invention are e.g. ménière's disease, acute peripheral vestibulopthy and tinnitus.

Thus in some embodiments the present invention provides a PPAR agonist for use in a method of preventing or inhibiting hair cell degeneration or hair cell death in a subject, wherein hair cell degeneration or hair cell death is related to and/or caused by ménière's disease, acute peripheral vestibulopthy and/or tinnitus.

In some embodiments the present invention provides a PPAR agonist for use in a method of preventing or treating ménière's disease in a subject.

In some embodiments the present invention provides a PPAR agonist for use in a method of preventing or treating acute peripheral vestibulopthy in a subject.

In some embodiments the present invention provides a PPAR agonist for use in a method of preventing or treating tinnitus in a subject.

Hearing Loss, Hair Cell Degeneration or Hair Cell Death Caused by a Noise Trauma or by Medical Intervention

Exposure to loud noise causes noise-induced hearing loss (NIHL) by damaging the organs of Corti. Damage by NIHL depends upon both the level of the noise and the duration of the exposure. Hearing loss may be temporary (temporary threshold shift, TTS) if a repair mechanism is able to restore the organ of the Corti. However, it becomes permanent (permanent threshold shift, PTS) when hair cells or neurons die. Structural modifications correlated to noise trauma are of two types: (1) mild damage of synapses and or hair cell stereocilia which can be repaired by cellular repair mechanisms and accounts for TTS and recovery and (2) severe damage inducing hair cell and neuronal apoptosis which can not be repaired by cellular repair mechanisms and accounts for PTS.

A noise trauma as referred herein is a noise which is sufficient to cause damage to the organs of corti, in particular a noise trauma causing temporary or permanent hearing loss. A noise trauma can be caused by exposure to a sound pressure level of e.g., at least 70 dB (SPL), at least 90 dB (SPL), at least 100 dB (SPL), at least 120 dB (SPL) or at least 130 dB (SPL). Hearing loss can also be caused by a medical intervention usually by a medical intervention in the ear e.g. by cochlea surgery such as cochlear implantation.

In some embodiments the PPAR agonist is administered before the subject is exposed to a noise trauma or medical intervention. In some embodiments, the PPAR agonist is administered after the subject is exposed to a noise trauma or medical intervention. In a particular embodiment the PPAR agonist is administered prior to cochlear surgery i.e. before the subject undergoes cochlear surgery.

Hearing Loss, Hair Cell Degeneration or Hair Cell Death Caused by Age

Hearing loss caused by age also referred in the literature as “age-related hearing loss” is the cumulative effect of aging on hearing. It is normally a progressive bilateral symmetrical age-related sensorineural hearing loss. The hearing loss is most marked at higher frequencies. There are four pathological types of hearing loss caused by age:

1) sensory: characterised by degeneration of organs of corti. 2) neural: characterised by degeneration of cells of spiral ganglion. 3) strial/metabolic: characterised by atrophy of stria vascularis in all turns of cochlea. 4) cochlear conductive: due to stiffening of the basilar membrane thus affecting its movement.

Hearing loss caused by age to be prevented or treated by the methods of the present invention is usually related to the first pathological type i.e. hearing loss characterised by degeneration of organ of corti. Thus in some embodiments the PPAR agonist is administered to the subject prior to degeneration of organ of corti, e.g. prior to damage or apoptosis of hair cells and/or prior to hair cell degeneration or hair cell death.

Chemically Induced Hearing Loss, Hair Cell Degeneration or Hair Cell Death

Hearing loss, hair cell degeneration or hair cell death can be induced chemically i.e. by a chemical agent e.g. by an antibiotic, a drug, a chemotherapeutic agent, heavy metals or organic agents. Antibiotics which may cause hearing loss include for example cephalosporins such as cephalexin (Keflex), cefaclor (Ceclor), and cefixime (Suprax); aminoglycosides such as gentamycin, tobramycin and streptomycin; macrolides, such as erythromycin, azithromycin (Zithromax) and clarithromycin; sulfonamides such as trimethoprim-sulfamethoxazole or tetracylines such as tetracycline, or doxycycline. In particular hearing loss, hair cell degeneration or hair cell death is effectively prevented or treated by the methods of the present invention in a subject exposed to gentamycin.

Chemotherapeutic agents e.g. anti-cancer agents which may cause hearing loss, hair cell degeneration or hair cell death include for example platinum-containing agents e.g. cisplatin, and carboplatin, preferably cisplatin. Drugs which may cause hearing loss, hair cell degeneration or hair cell death include for example furosemide, quinine, aspirin and other salicylates. Heavy metals which may cause hearing loss include for example mercury, lead. Organic agents which may cause hearing loss, hair cell degeneration or hair cell death include for example toluene, xylene, or styrene. In some embodiments the PPAR agonist is administered to the subject before the subject is exposed to a chemical agent, thereby preventing the subject from chemically induced hearing loss, hair cell degeneration or hair cell death. In some embodiments the PPAR agonist is administered to the subject after the subject is exposed to a chemical agent thereby treating the subject having chemically induced hearing loss, hair cell degeneration or hair cell death.

In a preferred embodiment, when hearing loss is caused by a noise trauma or is chemically induced, the PPAR agonist is administered to the subject prior to exposure of the subject to the noise trauma or to the chemical wherein at least 50%, preferably at least 60%, more preferably at least 70%, in particular at least 80%, more particular at least 90% of the cell damage of the hair cells caused by the noise trauma or the chemical agent is prevented.

In one aspect of the invention, the present invention provides a PPAR agonist for use in a method of preventing or inhibiting hair cell degeneration or hair cell death in a subject. In a further aspect of the invention the present invention provides a method of preventing or inhibiting hair cell degeneration or hair cell death in a subject, which method comprises administering to the subject a PPAR agonist. In some embodiments the PPAR agonist is administered to the subject in an amount that is sufficient to prevent or inhibit hair cell degeneration or hair cell death in the subject. In a further aspect the present invention provides the use of a PPAR agonist for the manufacture of a medicament for preventing or inhibiting hair cell degeneration or hair cell death in a subject.

In some embodiments hair cell degeneration or hair cell death in a subject is caused by a noise trauma, by age, a medical intervention, sudden hearing loss, or ischemic events such as ischemic injury, or is chemically induced wherein the chemical induction is caused by e.g. an antibiotic or a chemotherapeutic agent. Noise trauma, age, a medical intervention, sudden hearing loss, or ischemic events, or chemical induction can cause hair cell degeneration or hair cell death in a subject as described above for methods or preventing or treating hearing loss.

In some embodiments hearing loss, hair cell degeneration or hair cell death is caused by hair cell damage. In some embodiments the PPAR agonist is administered to the subject prior to identification of said hair cell damage i.e. prior to occurrence of hair cell damage. In a preferred embodiment when hair cell damage is caused by a noise trauma or is chemically induced, the PPAR agonist is administered to the subject prior to exposure of the subject to the noise trauma or to the chemical agent wherein at least 50%, preferably at least 60%, more preferably at least 70%, in particular at least 80%, more particular at least 90% of the cell damage of the hair cells caused by the noise trauma or the chemical agent is prevented. Identification/occurrence of hair cell damage is usually determined by evaluation of the state of the hair cells which can be easily accomplished as described above or as disclosed in the examples.

Pharmaceutical Compositions for Use in the Methods of the Invention

Provided herein are also pharmaceutical compositions that include a PPAR agonist and e.g. a pharmaceutically acceptable diluent, excipient, or carrier for use in the methods described herein. Thus in a further aspect, the present invention provides a PPAR agonist for use in a method of preventing or treating hearing loss in a subject, wherein the PPAR agonist is administered to the subject a pharmaceutical composition comprising the PPAR agonist and a pharmaceutically acceptable diluent, excipient, or carrier. Also provided by the present invention is a pharmaceutical composition comprising the PPAR agonist and a pharmaceutically acceptable diluent, excipient, or carrier for use in a method of preventing or treating hearing loss in a subject. In some embodiments the pharmaceutical composition is administered to the subject in an amount that is sufficient to prevent or treat hearing loss in the subject. In a further aspect the present invention provides a method of preventing or treating hearing loss in a subject, which method comprises administering to the subject a pharmaceutical composition comprising the PPAR agonist and a pharmaceutically acceptable diluent, excipient, or carrier. In some embodiments the pharmaceutical composition is administered to the subject in an amount that is sufficient to prevent or treat hearing loss in the subject. In a further aspect the present invention provides the use of a pharmaceutical composition comprising the PPAR agonist and a pharmaceutically acceptable diluent, excipient, or carrier for the manufacture of a medicament for preventing or treating hearing loss in a subject.

In a further aspect the present invention provides a PPAR agonist for use in a method of preventing or inhibiting hair cell degeneration or hair cell death in a subject, wherein the PPAR agonist is administered to the subject as a pharmaceutical composition comprising the PPAR agonist and a pharmaceutically acceptable diluent, excipient, or carrier. Also provided by the present invention are pharmaceutical compositions comprising the PPAR agonist and a pharmaceutically acceptable diluent, excipient, or carrier for use in a method of preventing or inhibiting hair cell degeneration or hair cell death in a subject. In some embodiments the pharmaceutical composition is administered to the subject in an amount that is sufficient to prevent or inhibit hair cell degeneration or hair cell death in the subject. In a further aspect the present invention provides a method of preventing or inhibiting hair cell degeneration or hair cell death in a subject, which method comprises administering to the subject a pharmaceutical composition comprising the PPAR agonist and a pharmaceutically acceptable diluent, excipient, or carrier. In some embodiments the pharmaceutical composition is administered to the subject in an amount that is sufficient to prevent or treat hearing loss in the subject. In a further aspect the present invention provides the use of a pharmaceutical composition comprising the PPAR agonist and a pharmaceutically acceptable diluent, excipient, or carrier for the manufacture of a medicament for preventing or inhibiting hair cell degeneration or hair cell death in a subject.

In some embodiments, the pharmaceutical compositions include other medicinal or pharmaceutical agents, diluent, excipients, carriers, adjuvants, such as preserving, stabilizing, wetting or emulsifying agents, solution promoters, salts for regulating the osmotic pressure, and/or buffers. Diluents are e.g. water, glycols, oils or alcohols. Carriers are e.g. starches or sugars. Excipients are e.g. surface-active substances, emulsifiers, stabilizers, preservatives, flavorings, or fillers.

In other embodiments, the pharmaceutical compositions also contain other therapeutic substances. Optionally, otoprotective agents, such as antioxidants, alpha lipoic acid, calcium, fosfomycin or iron chelators, to counteract potential ototoxic effects that may arise from the use of specific therapeutic agents or excipients, diluents or carriers are included in the pharmaceutical compositions.

In some embodiments, the pharmaceutical compositions include a dye to help enhance the visualization of the pharmaceutical composition when applied. In other embodiments, the pharmaceutical compositions also include one or more pH adjusting agents or buffering agents to provide an endolymph or perilymph suitable pH. Suitable pH adjusting agents or buffers include, but are not limited to acetate, bicarbonate, ammonium chloride, citrate, phosphate, pharmaceutically acceptable salts thereof or combinations or mixtures thereof. Such pH adjusting agents and buffers are included in an amount required to maintain pH of the composition between a pH of about 5 and about 9, in a preferred embodiment a pH between about 6.5 to about 7.5.

Modes of Administration and Treatment

Drugs delivered to the inner ear and/or to the middle ear have been administered systemically via oral, intravenous or intramuscular routes. The PPAR agonist or the pharmaceutical composition used in the methods described herein is usually administered orally, topically in the ear or by injection into the inner ear and/or into the middle ear, preferably by injection into the middle ear. For some routes of administration, e.g. for injection into the inner ear and/or into the middle ear a sustained release system can be used. In some routes of administration the penetration of the active ingredient is facilitated by transport enhancers as e.g. hyaluronic acid, DMSO. In some routes of administration, in particular when the PPAR agonist or the pharmaceutical composition is administered by injection into the inner ear and/or into the middle ear a tixotropic or thermogeling formulation is used to enable a painless administration and forming a gel or a high viscous composition ensuring prolonged and continuous release of the active ingredient into the inner ear and/or into the middle ear. In some routes of administration, in particular when the PPAR agonist or the pharmaceutical composition is administered as ear drops a formulation that enhances penetration through the skin leading to local PPAR activation in the ear region can be used.

The PPAR agonist or the pharmaceutical composition can be located in contact with the crista fenestrae cochlea, the round window, the tympanic cavity, the tympanic membrane, the auris media or the auris externa. In further or alternative embodiments, the PPAR agonist or the pharmaceutical composition can be administered on or near the round window membrane via intratympanic injection. In other embodiments, the PPAR agonist or the pharmaceutical composition are administered on or near the round window or the crista fenestrae cochleae through entry via a post-auricular incision and surgical manipulation into or near the round window or the crista fenestrae cochleae area. Alternatively, the PPAR agonist or the pharmaceutical composition is applied via syringe and needle, wherein the needle is inserted through the tympanic membrane and guided to the area of the round window or crista fenestrae cochleae. The PPAR agonist or the pharmaceutical composition is then deposited on or near the round window or crista fenestrae cochleae for localized treatment.

Preferably the PPAR agonist or the pharmaceutical composition as described herein is administered by intratympanic injection into the inner ear and/or into the middle ear, preferably into the middle ear. Intratympanic injection of therapeutic agents is the technique of injecting an agent behind the tympanic membrane into the middle and/or inner ear, preferably into the middle ear.

In one embodiment, the compositions described herein are administered directly onto the round window membrane via transtympanic injection. In another embodiment, the auris-acceptable compositions described herein are administered onto the round window membrane via a non-transtympanic approach to the inner ear. In additional embodiments, the composition described herein is administered onto the round window membrane via a surgical approach to the round window membrane comprising modification of the crista fenestrae cochleae.

In one embodiment the delivery system is a syringe and needle apparatus that is capable of piercing the tympanic membrane and directly accessing the round window membrane or crista fenestrae cochleae of the auris interna.

In some embodiments, the delivery device is an apparatus designed for administration of therapeutic agents to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446. U.S. patent application Ser. No. 08/874,208 describes a surgical method for implanting a fluid transfer conduit to deliver therapeutic agents to the inner ear. U.S. Patent Application Publication 2007/0167918 further describes a combined otic aspirator and medication dispenser for intratympanic fluid sampling and medicament application.

The PPAR agonist or the pharmaceutical composition described herein are useful in surgical procedures including, by way of non-limiting examples, cochlea surgery, labyrinthotomy, mastoidectomy, stapedectomy, endolymphatic sacculotomy or the like. In a preferred embodiment the PPAR agonist or the pharmaceutical composition as described herein is administered prior to surgical procedures in particular prior to cochlea surgery.

The PPAR agonist or the pharmaceutical composition described herein is administered for preventive and/or therapeutic treatments. Preventive treatments comprise prophylactic treatments. In preventive applications, the PPAR agonist or the pharmaceutical composition is administered to a subject suspected of having, or at risk for developing a disease, disorder or condition as described herein. In therapeutic applications, the PPAR agonist or the pharmaceutical composition is administered to a subject such as a patient already suffering from a disorder disclosed herein, in an amount sufficient to cure or at least partially arrest the symptoms of the disease, disorder or condition as described herein. Amounts effective for this use will depend on the severity and course of the disease, disorder or condition, previous therapy, the subject's health status and response to the drugs, and the judgment of the treating physician.

In the case wherein the subject's condition does not improve, the administration of the PPAR agonist or the pharmaceutical composition may be administered chronically, which is, for an extended period of time, including throughout the duration of the subject's life in order to ameliorate or otherwise control or limit the symptoms of the subject's disease or condition. In the case wherein the subject's status does improve, the administration of the PPAR agonist or the pharmaceutical composition may be given continuously; alternatively, the dose of drug being administered may be temporarily reduced or temporarily suspended for a certain length of time (i.e., a “drug holiday”).

Once improvement of the patient's otic conditions has occurred, a maintenance PPAR agonist or the pharmaceutical composition dose is administered if necessary. Subsequently, the dosage or the frequency of administration, or both, is optionally reduced, as a function of the symptoms, to a level at which the improved disease, disorder or condition is retained.

In some preferred embodiments the PPAR agonist or the pharmaceutical composition is administered by a single injection into the inner ear and/or into the middle ear, preferably by a single intratympanic injection into the inner ear followed by oral administration or by a single intratympanic injection into the middle ear followed by oral administration, which is preferred, or by administration as ear drops with penetration into the inner ear. Oral administration can be provided chronically, which is, for an extended period of time, including throughout the duration of the subject's life. In some embodiments after long term treatment, e.g. long term treatment using oral administration hearing capacity is increased based on a reactivation of hair cells from a resting state. In some embodiments after long term treatment, e.g. long term treatment using oral administration hearing capacity is increased based on an increase of the number of hair cells or hair cell function subsequent to PPAR activation.

The amount of the PPAR agonist to be administered will vary depending upon factors such as the particular compound, disease condition and its severity, according to the particular circumstances surrounding the case, including, e.g., the specific PPAR agonist being administered, the route of administration, the condition being treated, the target area being treated, and the subject or host being treated.

In some embodiments the PPAR agonist is administered to the subject in a dose that is below the dose needed for the treatment of diabetes using a PPAR agonist. In some embodiments the PPAR agonist is administered to the subject in a dose that is a factor of 8-20 fold lower than the top dose evaluated and tested for the treatment of diabetes, in particular a factor of 8-20 fold lower than the top dose evaluated and tested for the treatment of diabetes in human. The top dose evaluated and tested for the treatment of diabetes in human e.g for a PPAR gamma agonist such as pioglitazone is usually in the range from about 30-45 mg/day. In some embodiments at the PPAR dose used the side effects seen in treatment of diabetes are not present.

In some embodiments the PPAR agonist is administered to the subject in a dose that is below the active dose for antidiabetic or anti-dyslipidemic effect of the PPAR agonist, in particular a dose that is below the active dose for antidiabetic or anti-dyslipidemic effect of the PPAR agonist in human.

In some embodiments, a PPAR agonist, usually PPAR gamma agonists, PPAR alpha agonists and/or PPAR alpha/gamma dual agonists, preferably a PPAR gamma agonist, more preferably pioglitazone is administered in human orally in a dose of 0.05-30 mg/day, preferably 0.1-10 mg/day, more preferably 0.5-5 mg/day.

In some embodiments, the PPAR agonist, usually PPAR gamma agonists, PPAR alpha agonists and/or PPAR alpha/gamma dual agonists, preferably a PPAR gamma agonist, more preferably pioglitazone is administered in human topically in the ear usually in a concentration of 0.001% w/v to 10% w/v, preferably in a concentration of 0.005% w/v to 5% w/v, more preferably in a concentration of 0.01% w/v to 2% w/v. Usually 50 μl to 1 ml, preferably 1 ml of a solution containing the PPAR agonist is administered.

In some embodiments, the PPAR agonist, usually PPAR gamma agonists, PPAR alpha agonists and/or PPAR alpha/gamma dual agonists, preferably a PPAR gamma agonist, more preferably pioglitazone is administered in human by injection into the inner ear and/or into the middle ear at a concentration of 0.005% w/v to 10% w/v, preferably 0.01% w/v to 5% w/v per single injection. Usually 50 μl to 1 ml, preferably 1 ml of a solution containing the PPAR agonist is injected by single injection.

Methods of identification of patients who are suspected of having, or at risk for developing hearing loss, hair cell degeneration or hair cell death are also comprised by the present invention. In some embodiments patients who are suspected of having, or at risk for developing hearing loss, hair cell degeneration or hair cell death are identified by measurement of serum and/or plasma adiponectin levels, in particular the measurement of high molecular weight adiponectin levels. In some embodiments the monitoring of the treatment success and/or the identification of the subject e.g. the identification of the subject who is suspected of having, or at risk for developing hearing loss, hair cell degeneration or hair cell death, is achieved by measurement of serum and/or plasma adiponectin levels.

Kits/Articles of Manufacture

The disclosure also provides kits for preventing or treating hearing loss and/or preventing or inhibiting hair cell degeneration or hair cell death in a subject, preferably in human. Such kits generally will comprise one or more PPAR agonist or the pharmaceutical composition disclosed herein, and instructions for using the kit. The disclosure also contemplates the use of one or more PPAR agonist or the pharmaceutical composition disclosed herein, in the manufacture of medicaments for treating, abating, reducing, or ameliorating the symptoms of a disease, dysfunction, or disorder in a mammal, such as a human that has, is suspected of having, or at risk for developing hearing loss, hair cell degeneration or hair cell death.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein generally will comprise one or more PPAR agonist or the pharmaceutical composition disclosed herein and packaging materials. Examples of pharmaceutical packaging materials include, but are not limited to, blister packs, bottles, tubes, inhalers, pumps, bags, vials, containers, syringes, bottles, and any packaging material suitable for a selected composition and intended mode of administration and treatment.

### EXAMPLES

### Example 1

### Protection Against Antibiotic-Induced Hair Cell Damage

Organs of Corti were obtained from post-natal day 5 Sprague-Dawley rats and placed in organ culture. Gentamycin treatment resulted is 50-70% loss of hair cells after 48 h in culture. Pioglitazone co-treatment was protective, almost completely preventing gentamicin-dependent hair cell loss, and largely preserving organ morphology.

Methods

Animal Procedures

All animal procedures were carried out according to protocols approved by the Kantonales Veterinäramt, Basel, Switzerland. Postnatal day 5 (p5) Sprague-Dawley rats were used for the studies. Studies were performed in vitro, using organ of Corti (OC) explants from p5 animals. Animals were sacrificed and the cochleae carefully dissected to separate the organ of Corti from the spiral ganglion, stria vascularis and Reissner's membrane [Sobkowicz H M, Loftus J M, Slapnick S M. Acta Otolaryngol Suppl. 1993; 502:3-36].

Tissue Culture

OCs were harvested then placed in culture medium [Dulbecco's Modified Eagle Medium supplemented with 10% FCS, 25 mM HEPES and 30 U/ml penicillin (Invitrogen, Carlsbad, Calif., USA)] and incubated for 24 hours at 37° C. in an atmosphere of 95% O2/5% CO2. After that period, the culture medium was replaced with fresh medium containing no compound or 200 μM gentamycin alone or 200 μM gentamicin with either 2 or 10 μM pioglitazone, and incubated for a further 48 hours at 37° C. Ten OC explants were used for each treatment condition.

Hair Cell Counting

After incubation with compounds, the OCs were fixed in 4% paraformaldehyde, washed and then stained with a fluorescein (FITC)-conjugated phalloidin to detect inner and outer hair cells. After staining, the OCs were visualized and photographed using a fluorescence microscope (Olympus FSX100). Outer and inner hair cells were separately quantitated for the apical, basal, and middle turn of each organ of Corti. The values for each turn were averaged for the 10 OCs used for each condition. Significant differences between treatment groups in numbers OHC and IHC were determined using analysis of variance (ANOVA) followed by the least significant difference (LSD) post-hoc test (Stat View 5.0). Differences associated with P-values of less than 0.05 were considered to be statistically significant. All data are presented as mean±SD.

Results

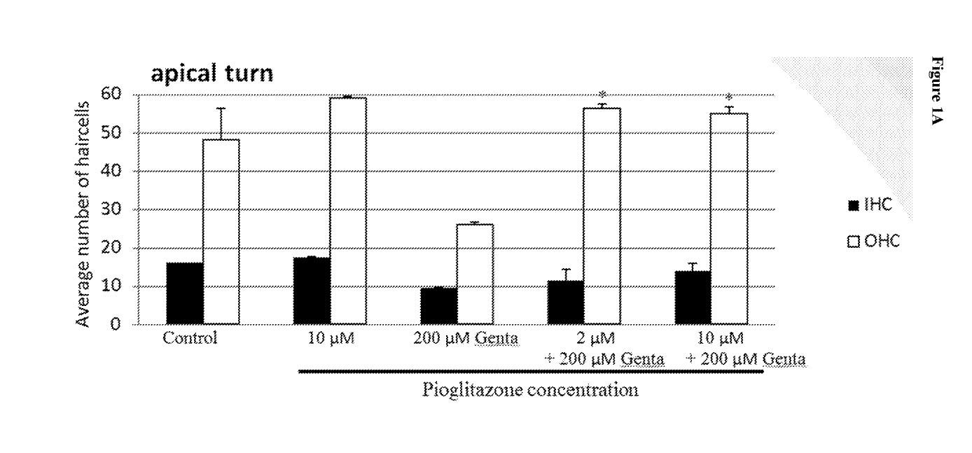

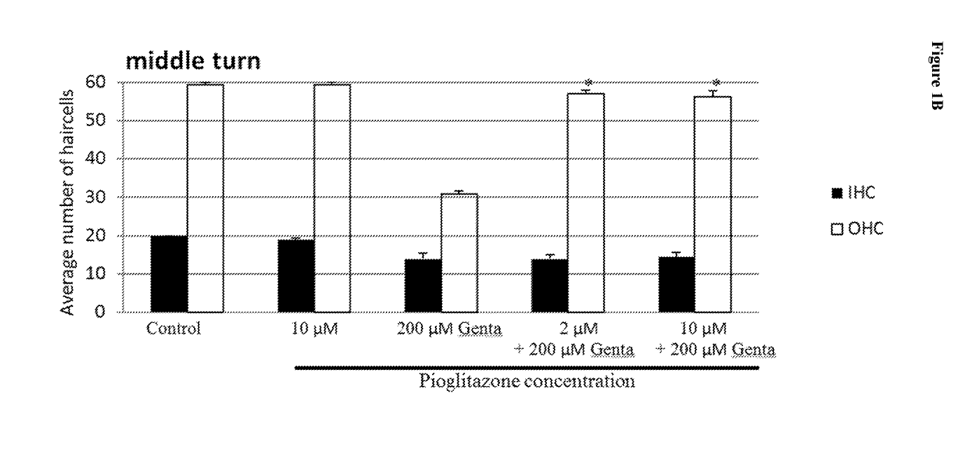

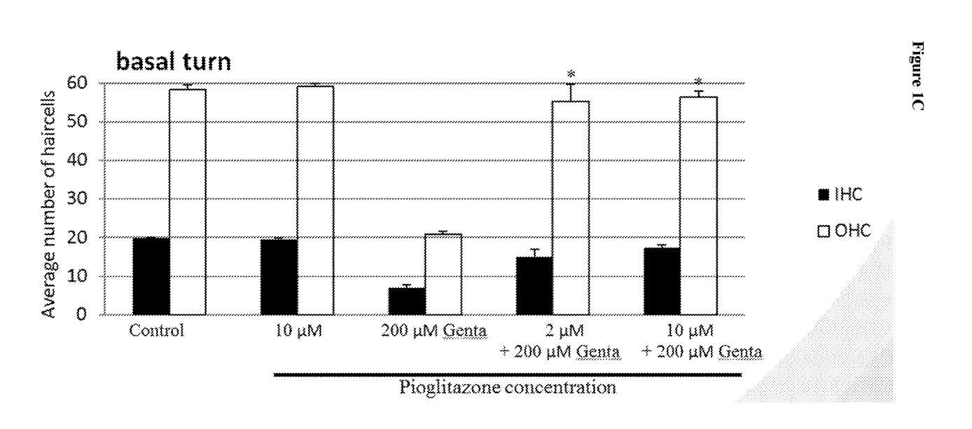

Untreated organs of Corti were well preserved after 48 hours in culture presenting with intact ordered rows of outer hair cells (OHC) and inner hair cells (IHC). Pioglitazone treatment alone, at either 2 or 10 μM had no effect on hair cell number or morphology, indicating no direct adverse effect of pioglitazone (FIG. 1A-C). In contrast, 200 μM gentamicin treatment resulted in almost complete destruction and loss of hair cells (FIG. 1A-C). Pioglitazone at both 2 and 10 μM was able to antagonize the effects of gentamicin and to preserve hair cell number and morphology (FIG. 1A-C). Quantative image analysis was performed to count IHC and OHC separately in the apical, basal, and middle turns of each organ of Corti. While gentamicin treatment resulted in a consistent reduction of hair cell number of approximately 50-70% in each segment, pioglitazone at both concentrations was able to completely prevent gentamicin-dependent hair cell loss in all turns.

### Example 2

### Protection Against Noise-Induced Hearing Loss

A formulation of pioglitazone or vehicle alone was applied into the middle ears of guinea pigs. The animals were then exposed to a noise trauma (broadband noise 4-20 kHz, 115 dB (SPL) and recording of hearing sensitivity over the standard frequency range was performed 7-14 days later. Results obtained in the hearing test were compared to baseline values before injury. Pioglitazone protected hearing, resulting in a reduction of >50% in the threshold shifts in pioglitazone-treated animals vs. vehicle controls.

Methods

Animal Procedures

The guinea pig model is the preferred animal species in hearing research. The agent application as well as the noise trauma was applied under general anesthesia. Upon arrival, animals underwent an acclimatization period of at least one week prior to experiments.

Animals were housed in pairs with ad libitum access to food and water in a temperature and humidity controlled environment on a 12 h/12 h light/dark cycle. The protocol was approved by the governmental animal use committee of Berlin, Germany.

Guinea pigs were first anaesthetized and hearing evaluated by a standard ABR method then into treatment groups. Each animal received a single round window application of test substance to both ears. The following day, animals were exposed to 115 dB broad band noise for 2 hrs under anaesthesia. At one and two weeks following noise exposure, the animals underwent a second hearing evaluation.

Animals were dosed with a single 40 μl application of pioglitazone formulation or matching vehicle, onto the cochlear round window in both ears the day prior to noise exposure. For this approach, a hole was drilled in the rostral part of the skull to directly access the Bulla which allows drug application to the round window under visual control.

Animals were noise-exposed in a soundproof chamber (0.8×0.8×0.8 m, minimal attenuation 60 dB) for 2 h to broadband white noise (5-20 kHz) at 115 dB sound pressure level (SPL) under anaesthesia (60 mg/kg ketamine and 6 mg/kg xylazine). Noise was delivered binaurally by loudspeakers (HTC 11.19; Visaton, Haan, Germany) placed above the animal's head. The speakers were connected to an audio amplifier (Tangent AMP-50; Aulum, Denmark) and a DVD player.

Hearing Assessment

At baseline before the noise exposure and on day 7 and 14 after noise, frequency-specific (2; 4; 8; 12; 16; 20; 24; 28; 32; 36; 40 kHz) auditory brainstem responses (ABR) were recorded in all treated animals and in controls. Auditory stimuli were delivered binaurally at different SPLs with a sinusoid generator (Model SSU2; Werk Fernmeldewesen, Berlin, Germany). Frequency output was controlled and adjusted with a digital counter (1941A Digital Counter; Fluke, Scarborough, Ontario, Canada). Sub-dermal needle electrodes were placed at the vertex (active), mastoid (reference), and in one foot (ground). ABR recordings were carried out with a Viking IV-measurement system (Viasys Healthcare, Conshohocken, Pa.). The brainstem responses were amplified (100,000×), filtered (bandpass 0.15-3 kHz), and averaged (300×) by the Viking IV-system. The amplitudes of the ABR waves were measured at different sound intensities by changing the attenuation of signal amplification. The amplitude-growth function was calculated for each tested frequency, and a linear regression was fitted to the linear portion of the data. The hearing threshold could be calculated for each frequency by extrapolating the linear amplitude-grow function of the regression line to zero. From these data, threshold differences (mean threshold shifts) were calculated between the control and the noise-exposed animals using the average values. Results are represented as mean relative hearing loss (±SD) in decibels (dB) of the experimental groups compared to controls.

Results

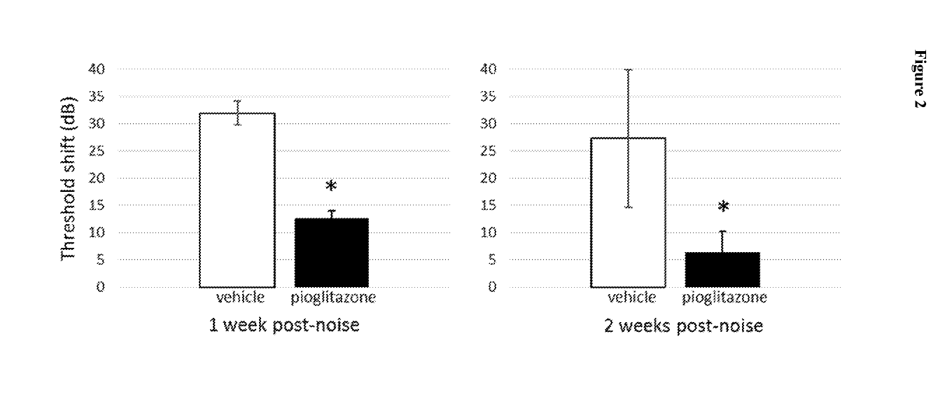

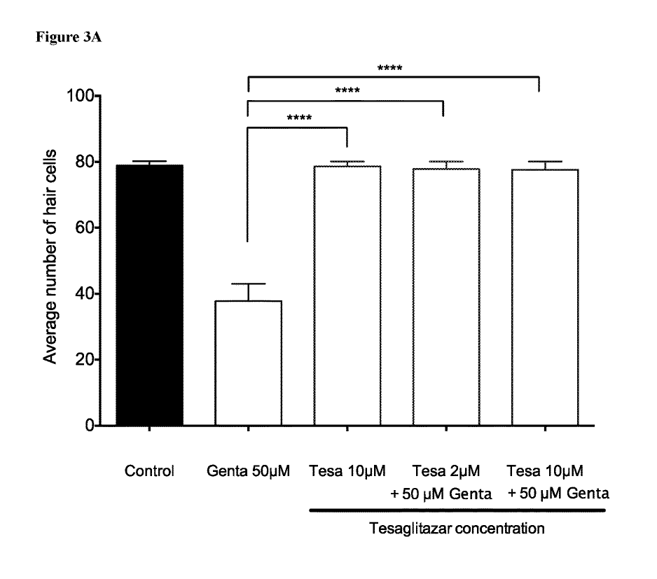

Vehicle treated animals showed a significant average hearing loss of 31.9±2.2 dB (mean±SD) over the frequency range of the noise challenge (5-20 kHz) at one week. Pioglitazone afforded significant protection of approximately 60% from noise-induced hearing loss, with only modest threshold shifts of 12.7±1.3 dB (mean±SD) (FIG. 2).

At two weeks, slight recovery in both groups was noted. Vehicle treated animals showed a significant average hearing loss of 27.3±12.6 dB (mean±SD) at two weeks. Pioglitazone treated animals showed only modest threshold shifts of 6.3±3.9 dB (mean±SD) at two weeks. (FIG. 2). These data demonstrate efficacy of pioglitazone to protect hearing.

### Example 3

### Protection Against Antibiotic-Induced Hair Cell Damage by Dual PPARα/γ Agonists and a PPARα-Selective Agonist

The experiment was carried out similarly to the experiments in example 1. Gentamicin treatment resulted is 50% loss of hair cells after 24 h exposure to mouse OC's in culture. Treatment with the dual PPARα/γ agonists muraglitazar and tesaglitazar and with the PPARα-selective agonist fenofibric acid protected from gentamicin-dependent hair cell loss.

Methods

Methods were similar to those in Example 1. The main differences were that mouse OC's were used rather than rat OC's. Moreover, treatment was performed for 24 hrs with 50 μM gentamicin. The number of OC's used for each experimental condition was 3-5. The concentrations of test substances were 2 μM and 10 μM for tesaglitazar and muraglitazar, and 25 μM and 150 μM for fenofibric acid.

Results

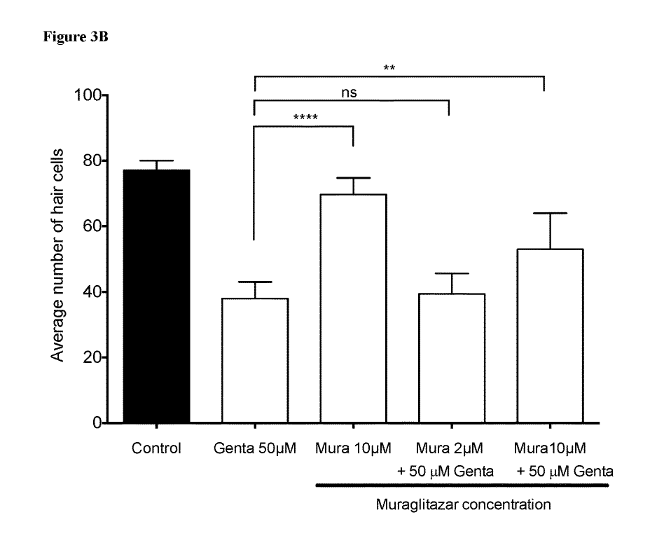

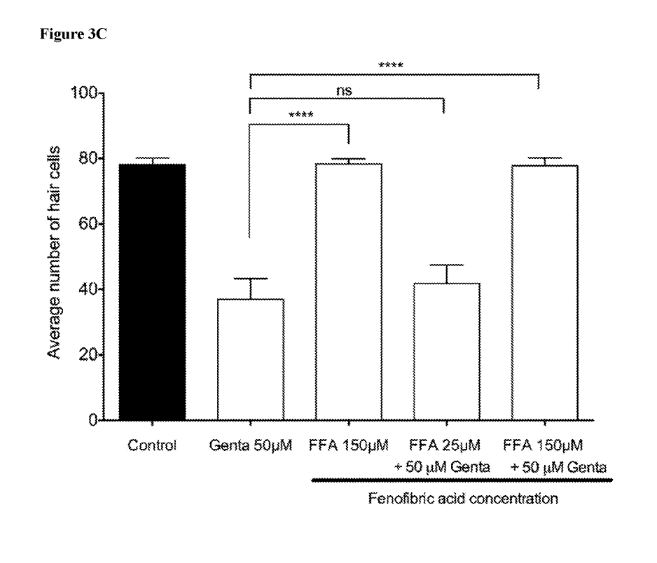

Untreated organs of Corti were well preserved after 24 hours in culture presenting with intact ordered rows of outer hair cells (OHC) and inner hair cells (IHC). None of the test substances alone and any concentration had an effect on hair cell number or morphology, indicating no direct adverse effects (FIG. 3A-C). In contrast, 50 μM gentamicin treatment resulted in approximately 50% loss of hair cells (FIG. 3A-C). Tesaglitazar at both 2 and 10 μM was able to antagonize the effects of gentamicin and to preserve hair cell number and morphology (FIG. 3A). Muraglitazar was not effective at 2 μM but was partially protective at 10 μM (FIG. 3B). Fenofibric acid was not effective at 25 μM but was completely protective at 150 μM (FIG. 3C).

1. A PPAR agonist for use in a method of preventing or treating hearing loss in a subject.

2. A PPAR agonist for use in a method of preventing or inhibiting hair cell degeneration or hair cell death in a subject.

3. The PPAR agonist for use in a method of claim 1, wherein the PPAR agonist is activating PPAR alpha, PPAR gamma, or PPAR delta or combinations thereof.

4. The PPAR agonist for use in a method of claim 1, wherein the PPAR agonist is activating PPAR gamma.

5. The PPAR agonist for use in a method of claim 1, wherein the PPAR agonist is pioglitazone.

6. The PPAR agonist for use in a method of claim 1, wherein hearing loss, hair cell degeneration or hair cell death is caused by hair cell damage.

7. The PPAR agonist for use in a method of claim 1, wherein hearing loss is sudden hearing loss.

8. The PPAR agonist for use in a method of claim 1, wherein hearing loss is caused by a noise trauma, by a medical intervention, by ischemic injury, by age or is chemically induced.

9. The PPAR agonist for use in a method of claim 1, wherein hearing loss is caused by a noise trauma or is chemically induced and the PPAR agonist is administered to the subject prior to exposure of the subject to the noise trauma or to the chemical agent and wherein at least 50% of the cell damage of the hair cells caused by the noise trauma or the chemical agent is prevented.

10. The PPAR agonist for use in a method of claim 1, wherein the PPAR agonist is administered prior to cochlea surgery.

11. The PPAR agonist for use in a method of claim 1, wherein the PPAR agonist is administered orally, topically in the ear, by injection into the inner ear and/or by injection into the middle ear.

12. The PPAR agonist for use in a method of claim 1, wherein the PPAR agonist is administered by intratympanic injection into the middle ear.

13. The PPAR agonist for use in a method of claim 1, wherein the PPAR agonist is administered by a single injection into the inner ear and/or into the middle ear followed by oral administration or followed by administration as ear drops with penetration into the inner ear.

14. The PPAR agonist for use in a method of claim 1, wherein the PPAR agonist is administered to the subject in a dose that is below the dose needed for the treatment of diabetes using said PPAR agonist.

15. The PPAR agonist for use in a method of claim 1, wherein the subject is a human and the PPAR agonist is administered orally in a dose of 0.5-5 mg/day, topically in the ear in a dose of 0.01% to 2% or by injection into the inner ear and/or into the middle ear at a concentration of 0.01% to 5% per single injection.

16. The PPAR agonist for use in a method of claim 1, wherein the PPAR agonist is administered to the subject as a pharmaceutical composition comprising the PPAR agonist and a pharmaceutically acceptable diluent, excipient, or carrier.

17. A pharmaceutical composition comprising a PPAR agonist and a pharmaceutically acceptable diluent, excipient, or carrier for use in a method of preventing or treating hearing loss in a subject or for use in a method of preventing or inhibiting hair cell degeneration or hair cell death in a subject.

18. A kit for preventing or treating hearing loss and/or preventing or inhibiting hair cell degeneration or hair cell death in a subject comprising a PPAR agonist or a pharmaceutical composition comprising a PPAR agonist and a pharmaceutically acceptable diluent, excipient, or carrier, and instructions for using the kit.
